(19) United States
(12) Patent Application Publication
(10) Pub. No.: US 2025/0084117 A1
(43) Pub. Date: Mar. 13, 2025

(54) SPACED HEPARINOIDS: A NEW CLASS OF HEPARIN-LIKE MOLCULES

(71) Applicants: Sourena Nadji, St. Louis, MO (US); Yingjie Ren, St. Louis, MO (US); Linfeng Fan, San Mateo, CA (US); Raj Dhar, Ballwin, MO (US)

(72) Inventors: Sourena Nadji, St. Louis, MO (US); Yingjie Ren, St. Louis, MO (US); Linfeng Fan, San Mateo, CA (US); Raj Dhar, Ballwin, MO (US)

(73) Assignee: PharmaRen Discovery, LLC, Berkeley, MO (US)

(21) Appl. No.: 18/632,623

(22) Filed: Apr. 11, 2024

Related U.S. Application Data
(60) Provisional application No. 63/581,141, filed on Sep. 7, 2023.

(51) Int. Cl. C07H 15/12; C07H 1/00

(52) U.S. Cl. CPC C07H 15/12; C07H 1/00

(57) ABSTRACT

This invention relates to compositions comprising heparin-like oligosaccharides with defined structures that are covalently connected together at a precise distance from each other by flexible linkers, and to the method for the manufacture of said oligosaccharides. Particularly this invention relates to the design and the method of manufacturing of ‘spaced-heparinoids’ of Formulas 1 and 2, wherein S¹, S², S³, S⁴, and S⁵ independently comprise mono- or oligosaccharides sequences connected by length-adjustable linkers, L¹, L², and L³. The spaced heparinoids are synthesized

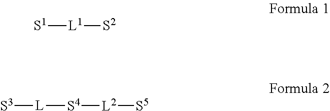

from short sequences of synthetic oligosaccharides with varying sulfation patterns which are covalently linked to each other through aliphatic linkers. The present invention also discloses a new method of manufacture that will generate non-polydisperse and linear head-to-tail heparinoids that are linked to each other through a “isocyanate-amine” amide bond forming chemistry.

This application claims benefit of priority based on provisional application No. 63/581,141 filed on Sep. 7, 2023, and said application is incorporated herein by reference in its entirety.

### CROSS-REFERENCE TO RELATED APPLICATIONS

Not applicable.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### INCORPORATION-BY-REFERENCE OF MATERIAL SUBMITTED ON A COMPACT DISC

Not Applicable.

### BACKGROUND OF THE INVENTION

### Field of the Invention

This invention relates to compositions comprising heparin-like oligosaccharides with defined structures that are covalently connected together at a precise distance from each other by flexible linkers, and to the method for the manufacture of said oligosaccharides.

### Description of the Related Art

Various prior art references in the specification are indicated parenthetically by author and year. Full citation corresponding to each reference is listed alphabetically at the end of the specification and is herein incorporated by reference in its entirety to describe fully and clearly the state of the art to which this invention pertains. Unless otherwise specified, all technical terms and phrases used herein conform to standard organic and medicinal chemistry nomenclature established by International Union of Pure and Applied Chemistry (IUPAC), the American Chemical Society (ACS), and other international professional societies. The rules of nomenclature are described in various publications (Fox and Powell, 2001; Hellwinkel, 1992), which are herein incorporated by reference in their entireties.

Heparan sulfate (HS) proteoglycans (PGs) are expressed on cell surfaces and in the extracellular matrix of most animal tissues and interact with numerous proteins responsible for critical physiological and pathophysiological events (Weiss et al., 2017; Xu & Esko, 2014). The synthesis of HS starts from the polymerization of repeating disaccharides of N-acetylglucosamine and glucuronic acid in the Golgi apparatus that in some cases reaches up to 50 disaccharides or more. Through the engagement with glycosyl polymerase enzymes EXT1 and EXT2, polymerizes to a chain containing 20-25 heterodimeric complexes. The chain is further modified by interacting with other enzymes, such as the family of N-deacetylase-N-sulfotransferase that removes the acetyl group of some GlucNAc residues and substitute them with the sulfate group. Interactions of the HS chain with O-sulfotransferase family of enzymes produce O-sulfation at positions C2, C6, or C3 around glucosamine in an irregular pattern. In addition, an epimerase enzyme is involved that epimerizes specific D-glucuronic acid residues to L-iduronic acid. The variable/irregular actions of these modifying enzymes give rise to heterogeneous and polydisperse HS chains with partly sulfated domains (so-called NS domains), which are separated from NA domains that are lacking all or most of the sulfate groups. The mixed NS/NA segment is located in between. These features of the HS chain represent the structural basis for the ability of HS to strongly and specifically interact with proteins (HS binding proteins, HSBP). The protein domain(s) responsible for interactions with HS is (are) involved in two binding motifs, XBBXBX and XBBBXXBX, where B represents a basic residue and X represents a noncharged residue. However, the structural basis of HS interactions with proteins has not ben fully elucidated. The consensus is that, except in rare cases such as antithrombin, most HSBP interact with more than one sequence in HS, and the interacting sequences are located at specific distances from each other (Muñoz & Linhardt, 2004) (FIG. 1). Understanding these as well as interruption of this mode of HS/HSBP interactions is essential in discovering new HS-based drugs and potential application in the treatment of diseases such as cancer and cognitive disorders. Currently, molecules capable of presenting multi-oligosaccharide domains to the HSBP are limited. Chemoenzymatic and semi-synthetic approaches have provided access to complex oligosaccharide molecules (Liao et al., 2023). However, general applications of such methods have severe limitations, such as access to specific enzymes, which are now in the hands of very few laboratories. Some innovative research groups have disclosed more accessible approaches, and those are based on short oligosaccharides on “polymeric” or “dendritic” platforms (Brown et al., 2012; Griffin & Hsieh-Wilson, 2013; Loka et al., 2019; Oh et al., 2013; Sheng et al., 2013; Tyler et al., 2015; Zubkova et al., 2018). In the polymeric form, polymeric scaffolds are constructed through ring-opening polymerization (ROP) of monomers that are linked to short oligosaccharides (1-4 mers) (Loka et al., 2019). In the dendritic form, specific small oligosaccharides are attached to the outer shell of dendrimers. Some of these polymers and dendrimers have displayed notable biological activities and demonstrated the importance of a positive, cooperative binding mode (Zubkova et al., 2018). However, in these prior art technologies, they do not disclose any compositions comprising multiple linear stretches of well-defined sulfated oligosaccharide sequences of variable lengths at a precise distance from each other (like ‘beads-on-a-string’). Because of their potential impact on the affinity and specificity of HS-protein interactions, such ‘spaced’ heparinoids will be of considerable importance. However, this class of “spaced” heparinoids cannot be prepared using the prior art methods because they generate polydisperse polymers or non-linear heparinoids, and they cannot be obtained from natural HS or heparin. Therefore, new synthetic methods that are practical, reproducible, and scalable are needed. There are two recent publications that address both issues of linearity and polydispersity of the earlier approaches. Both methods link oligosaccharides through aliphatic linkers. The linking chemistry in one method is achieved through click chemistry, and in the other through the classical peptide coupling chemistry. Both methods link the heparinic disaccharide or tetrasaccharide from their reducing ends and through anomeric [ethylamine glycosidic] linkage to the C-4 of the non-reducing end in a linear head-to-tail fashion.

In the present invention, however, we disclose new heparinoids and the method of manufacture of said heparinoids that will generate non-polydisperse and linear head-to-tail molecules that are linked to each other through a unique “isocyanate-amine” amide bond forming chemistry. The chemistry is simple, and at pH 9-10, the coupling produces nearly quantitative yield of the product. Unlike the prior art method involving click and peptide coupling reactions, the methods disclosed in the present invention are not substrate-dependent and has minimum variabilities in the yield. The clean conversion makes the separation and purification much easier, and this is a concern specially when the lengths of the spaced heparinoids grow longer and multiple tetra-, penta-, and higher number of the saccharides are involved. Very importantly, because of the methods of covalent connection of the oligosaccharides of the present invention is directed away from the anomeric centers, the spaced heparinoids of the present invention are mostly free of possible α/β isomeric contaminations. Further, and equally important, the current invention allows the linking operation to be performed both on the fully protected oligosaccharides (in organic solvents) and on the fully deprotected, hydrogenated, and sulfated oligomeric heparinoids (in aqueous media).

### SUMMARY OF THE INVENTION

The present invention introduces spaced heparinoids which are synthesized from short sequences of synthetic oligosaccharides with varying sulfation patterns. They are covalently linked to each other through aliphatic linker(s) (FIG. 2). Thus, invention particularly relates to the ‘spaced-heparinoids’ compositions and the method of manufacturing said heparinoids.

Particularly the present invention relates to spaced-heparinoids of Formulas 1 and 2, wherein S¹, S², S³, S⁴, and S⁵ independently comprise mono-or oligosaccharides sequences connected by length-adjustable linkers, L¹, L², and L³. The present invention describes the design and the method of manufacturing of spaced-heparinoids of Formulas 1 and 2.

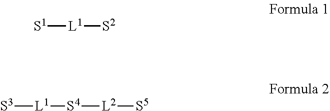

### BRIEF DESCRIPTION OF FIGURES

FIG. 1. Cartoon representation of HSBP interactions with HS domains heparinoids.

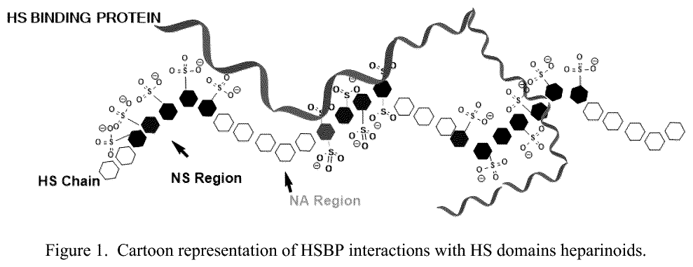

FIG. 2. Schematic illustration of beads-on-a-string or spaced-heparinoids.

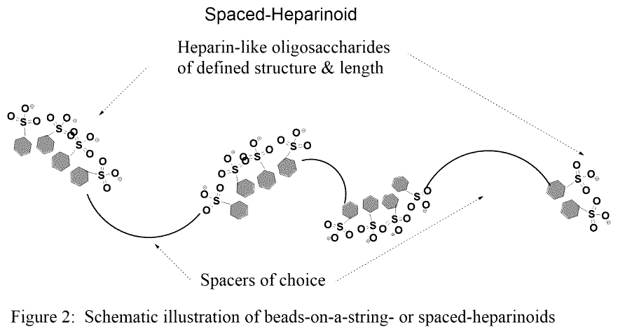

FIG. 3. Commercial building blocks for the synthesis of spaced-heparinoids.

FIG. 4. NMR spectra of compound 27 (5+3+5).

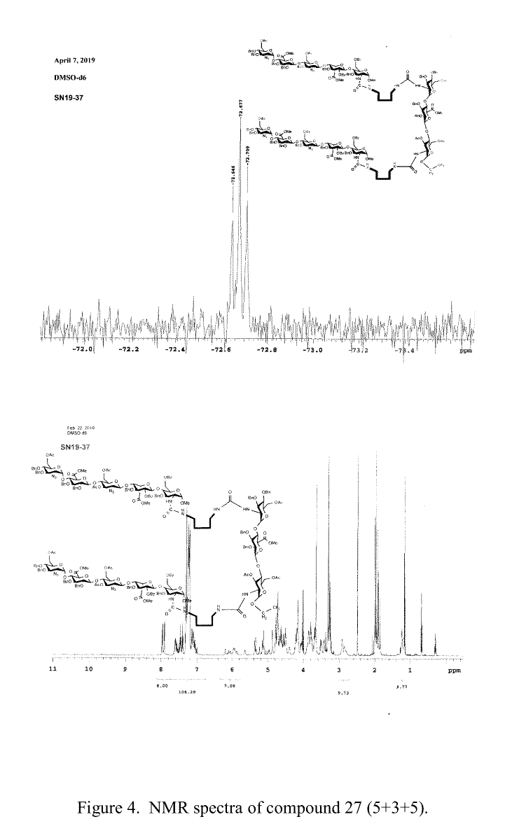

FIG. 5. NMR spectra of compound 28 (1+3+1).

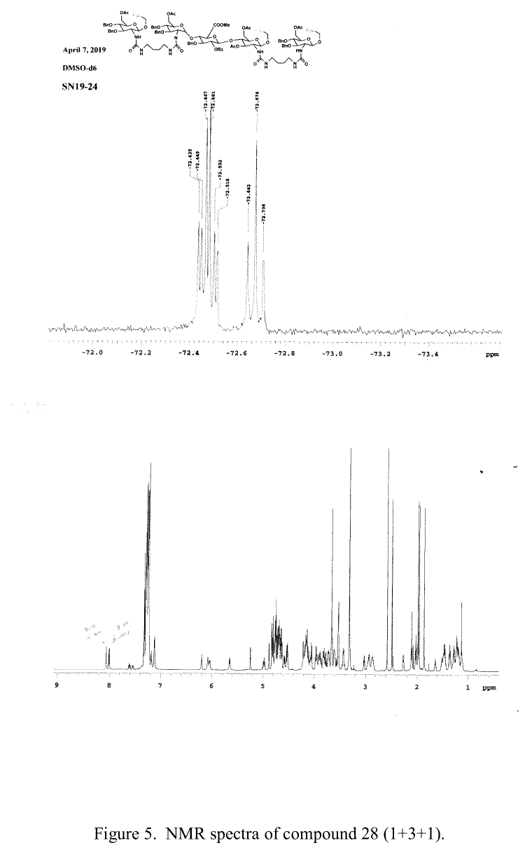

FIG. 6. NMR spectrum of compound 24.

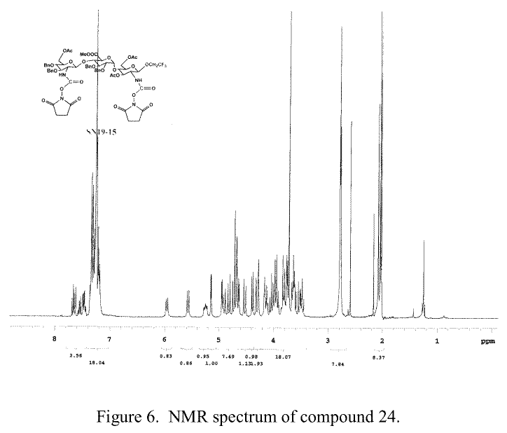

FIG. 7. NMR spectrum of compound 23i.

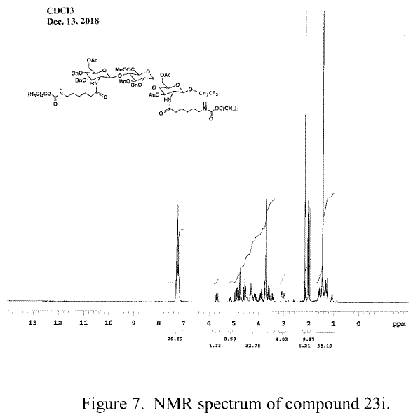

FIG. 8. NMR spectrum of compound 22.

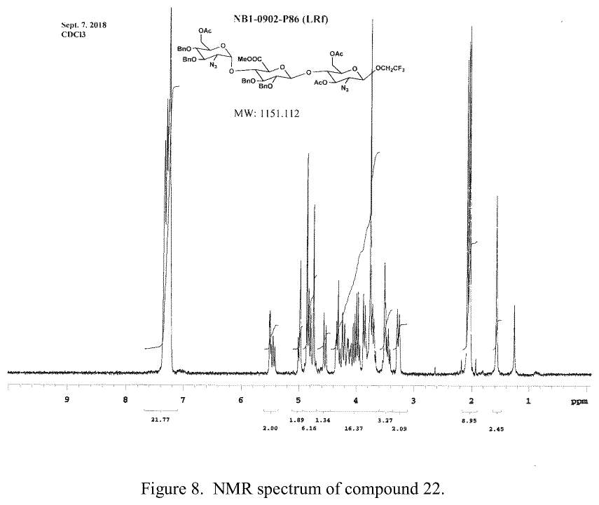

FIG. 9. NMR spectrum of compound 19.

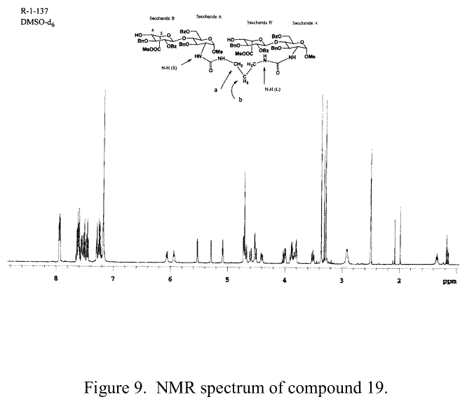

FIG. 10. NMR spectrum of compound 20.

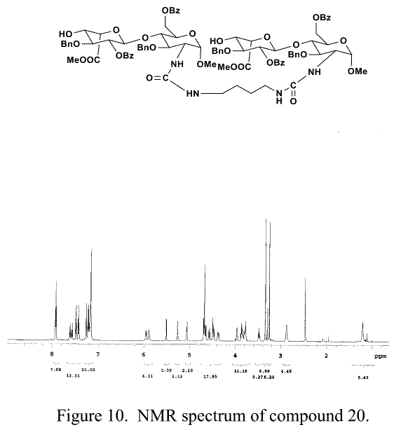

FIG. 11. NMR spectrum of the pentasaccharide with amino linker.

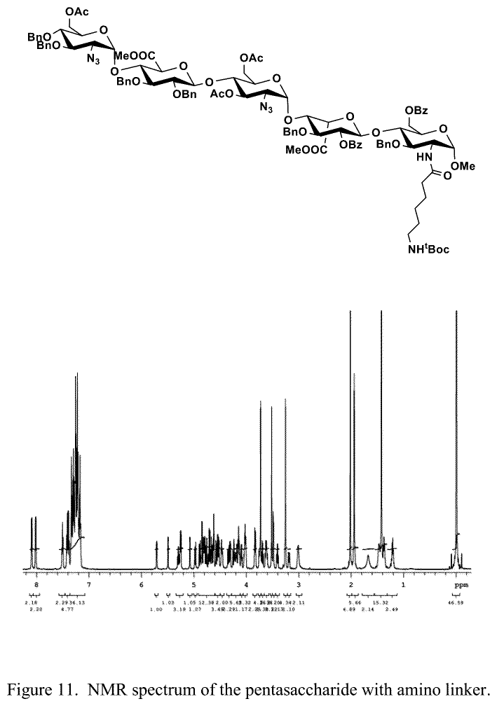

FIG. 12. NMR spectrum of spaced heparinoids of type 3+3+3.

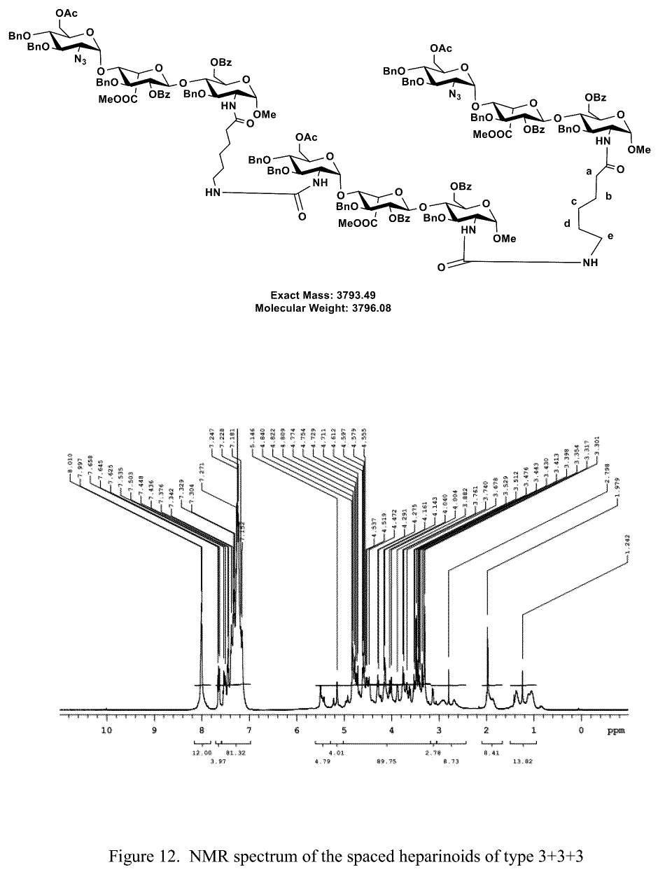

### DETAILED DESCRIPTION

The present invention particularly relates to ‘spaced heparinoids’ of Formulas 1 and 2,

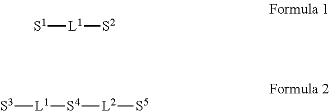

wherein S¹, S², S³, S⁴, and S⁵ independently mono-or oligosaccharides comprising:

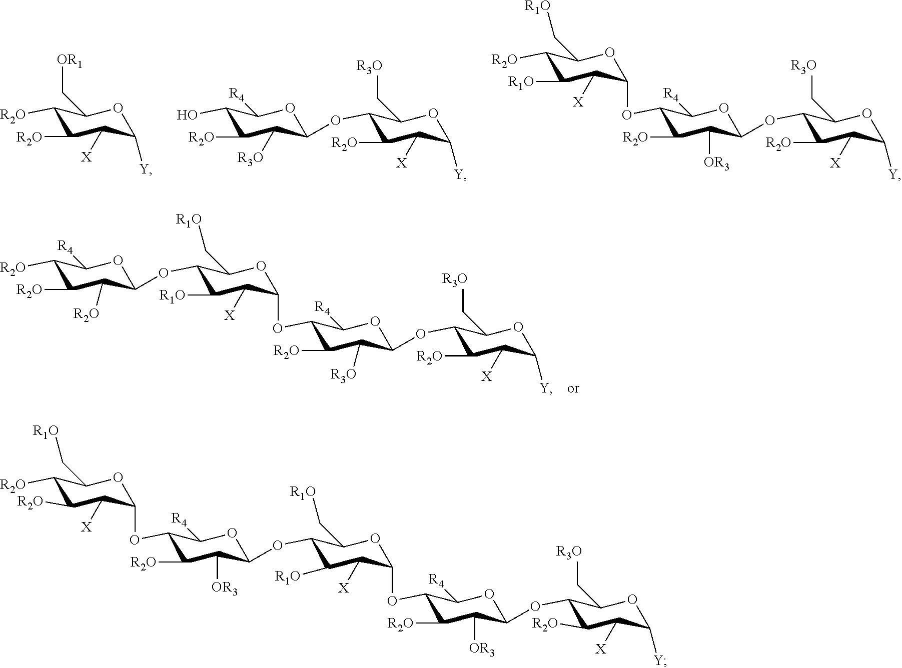

L, L¹, and L² are independently length-adjustable spacers that covalently connect said mono-or oligosaccharides, and are independently selected from the group consisting of

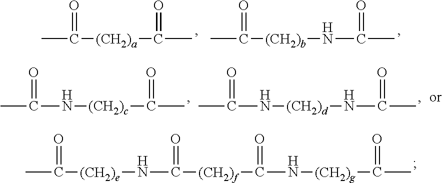

Each of X and Y; and each of R¹, R², R³, and R⁴ may be the same or different, and are independently selected from the group comprising the substituents listed in Table 1 below.

TABLE 1
Substituents at the oligosaccharides.

| | |
|---|---|
| X | N₃, NH₂, NHAc, NHSO₃Na, —NH—, or —NCO. |
| Y | —OH, —OMe, —OCH₂CF₃, or C₅-C₁₀ —OAryl |
| R¹ | —CH₂OH, C₁-C₁₀ Alkyl, —COOMe, or —COOBn. |
| R² | Bn or PMB. |
| R³ | C₁-C₁₀ Acyl, or Bz. |
| R⁴ | —COOH, —COONa, —COOMe or —COOBn. |
| R⁵ | C₁-C₁₀ Acyl, or Bz. |

Each of the subscripts ‘a’ to ‘g’ independently vary from 1-10.

One embodiment of the present invention, as illustrated by structure 1, comprises Type 1+1 oligosaccharide compositions of Formula 1, wherein two monosaccharides are connected by the spacer L¹,

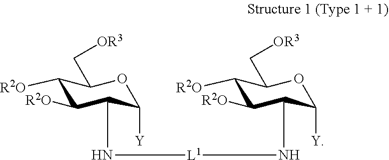

Another embodiment of the present invention, as illustrated by structure 2, comprises Type 1+2 oligosaccharide compositions of Formula 1, wherein X is —NH—, and the monosaccharide and the disaccharide are connected by the spacer L¹,

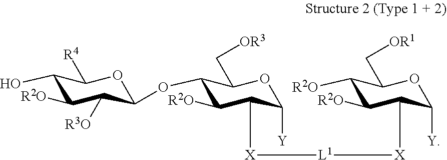

Another embodiment of the present invention, as illustrated by structure 3, comprises Type 2+2 oligosaccharide compositions of Formula 1, wherein X is —NH—, and two disaccharides are connected by the spacer L¹,

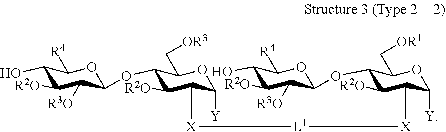

Another embodiment of the present invention, as illustrated by structure 4, comprises Type 1+3+1 oligosaccharide compositions of Formula 2, wherein X is —NH—, and a monosaccharide, a trisaccharide, and a monosaccharide are connected by the spacer L¹,

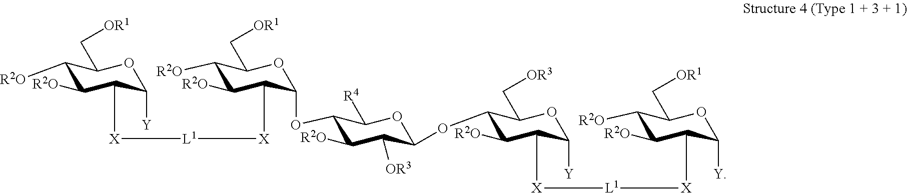

Another embodiment of the present invention, as illustrated by structure 5, comprises Type 2+3+2 compositions of Formula 2, wherein X is NH and a disaccharide, a trisaccharide, and a disaccharide are connected by the spacers L¹ and L²,

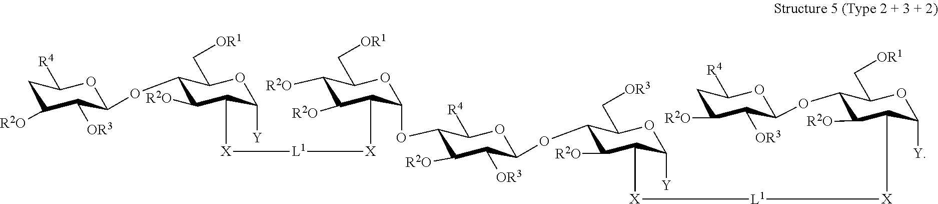

Another embodiment of the present invention, as illustrated by structure 6, comprises Type 4+4 compositions, wherein two tetrasaccharides are connected by the spacer L,

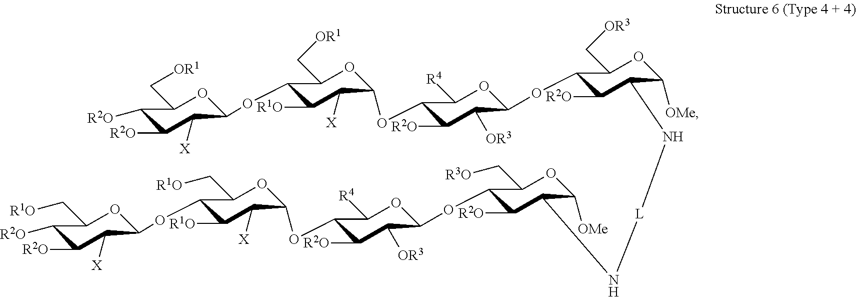

Another embodiment of the present invention, as illustrated by structure 7, comprises Type 5+5 compositions wherein two pentasaccharides are connected by the spacer L,

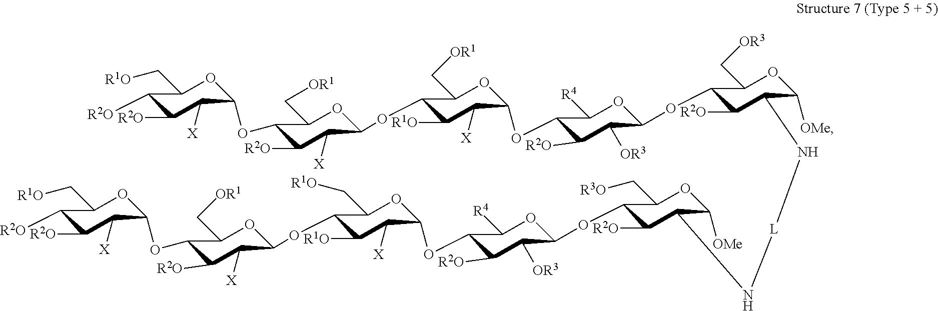

Another embodiment of the present invention, as illustrated by structure 8, comprises Type 5+5 compositions, wherein two pentasaccharides are connected by the spacer L¹ and wherein L¹ is —CO(CH₂)₆CO—, and X is —N₃, —NH₂, or —NHSO₃Na.

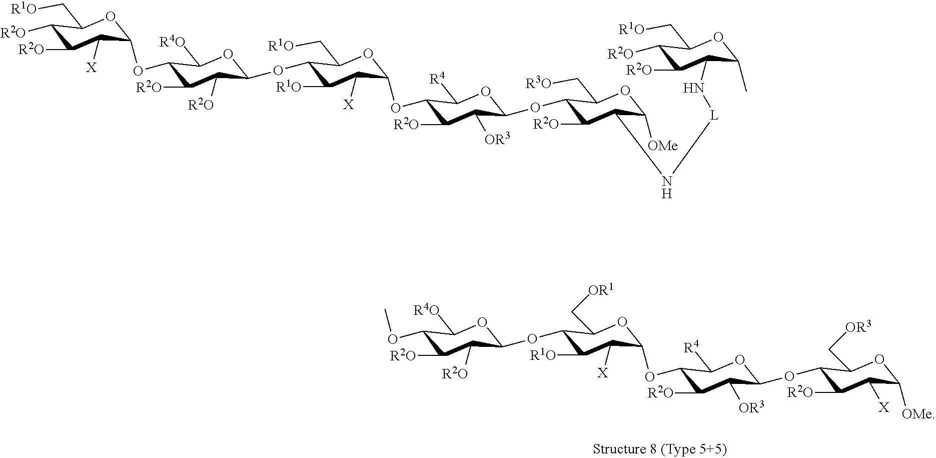

Another embodiment of the present invention, as illustrated by structure 9, comprises Type, 5+3+5 compositions of the following Formula, wherein a pentasaccharide, a trisaccharide, and a pentasaccharide are connected by the spacer L¹, and wherein L¹ is —CONH(CH₂)₄NHCO—,

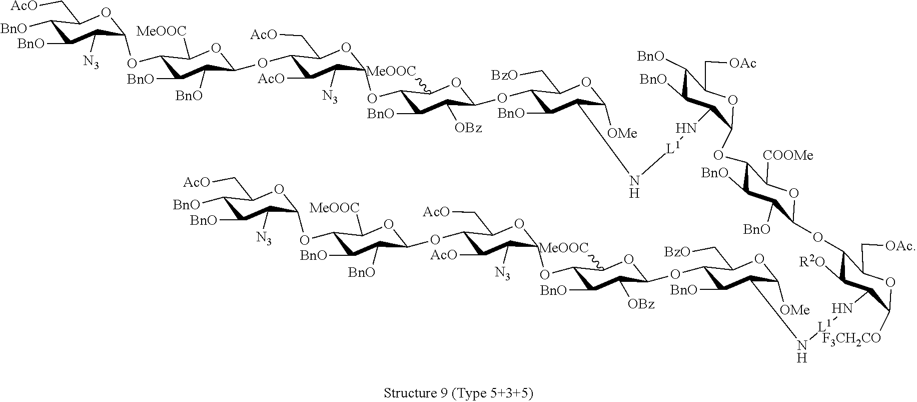

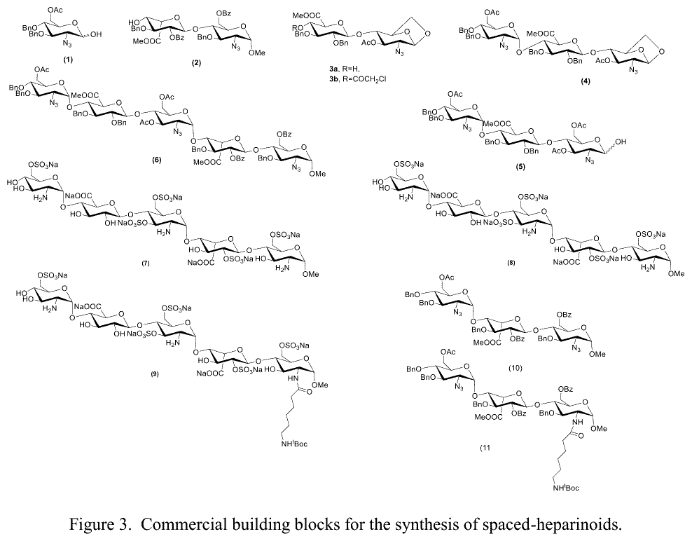

The sugar building blocks of the present invention are mostly available commercially [www.heparinbuildingblocks.com] or can be prepared by known methods in carbohydrate chemistry (Avci et al., 2003; Bonnaffé, 2011; Walenga et al., 2002). However, converting the short oligosaccharides to “spaced heparinoids” requires the new chemistry described in this invention. Commercially available starting materials for the preparation of mono-and oligosaccharide building blocks are depicted in FIG. 3. The linking of mono-or oligosaccharide units primarily achieved through forming amide bonds. Among the literature of amide bond formation methods, the amine reaction with isocyanates is the most efficient. Treatment of amino sugars with di(N-succinimidyl) carbonate generates the corresponding isocyanate derivatives. The reaction is facile, nearly quantitative, and presents no complications from any side reactions.

The amino-sugar precursor was obtained from the corresponding azido-derivatives through triphenylphosphine or trimethyl phosphine (Staudinger reaction) or reduction via Zn/ammonium chloride treatment. The oligosaccharide building blocks with amino-linkers of different lengths and structures were prepared by bifunctional diamines such as 1,3-diamino propane, 1,4-diamino butane, and 1,6-diaminohexane which are protected at one end with N-hydroxy succinimidyl carbamoyl or t-Boc. By utilizing building blocks containing the isocyanate group and the building blocks carrying amino linkers, a variety of “spaced” oligosaccharides were prepared. The methodology described here can produce molecules of well-defined domains and spacing. The strategy of linking the building blocks via the amide bonds not only offers chemically stable molecules but produces them in almost quantitative yields and with no isomerization problem that is commonly encountered in the prior art involving connecting saccharides through glycosylation.

The general procedure for the manufacture of spaced-heparinoids of the present invention are described in the following paragraphs below. It may be noted that all reagents were obtained commercially and used as received unless specified otherwise. Solvents used for chromatography were ACS or HPLC grade, as appropriate.

The Formation of Trichloroacetimidates: To the solution of the saccharide in dichloromethane (0.02-0.2 M solution) at 0-5° C. was added trichloroacetonitrile (1.4 equiv.) followed by DBU (0.14 equiv.). The reaction mixture stirred at 0° C. for 4-6 h or till TLC examination shows >95% completion. The reaction mixture was extracted with DCM/sat. NaCl and the product was purified on silica gel column.

Glycosylation: Acetimidate derivative of saccharide (mono-di- or trisaccharide) with reactant (saccharide or n-alkyl/fluoroalkyl alcohol), 1.3 equiv, were combined in a flask, co evaporated with toluene (3×10 mL). The anhydrous materials were than dissolved in Dichloromethane (DCM) to maintain a concentration of 0.02-0.05M (based on the Imidate). Reaction mixture was stirred at RT, and TMSOTf (0.1 equiv.) was added to the mixture under Argon atmosphere. Stirring was continued until TLC indicated disappearance of Imidate (30 min). Reaction mixture was extracted and purified by Silica Gel column chromatography.

Reduction of Saccharide Azide to Amine, Zn/Ammonium Chloride Method: Azido saccharide was dissolved in THF (0.01M) at room temperature. Zinc powder was added (8 equiv./azide) and mixture was cooled in an ice bath. Ammonium chloride solution (1M) 12 equiv./azide was added dropwise in 15 min. Ice bath was removed and reaction mixture was vigorously stirred overnight at RT, which resulted in a white-grey suspension liquid. The mixture was evaporated to dryness and extracted with EtOAc/dIH₂O Product was purified by Silica Gel column chromatography.

Reduction of Saccharide Azides to Amine using PPh₃: Azido saccharide was dissolved in THF (0.05M) and heated at 50° C. Triphenylphosphine, (2 equiv./Azide reduction) was added in one portion at 50° C. After 2 h, diH2O (230 equiv.) was added and the reaction mixture was stirred at RT overnight. Reaction mixture was extracted by Ethyl Acetate and the product was purified by Silica Gel chromatography.

Reaction of Amino sugars with Disuccinimidyl Carbonate: Amino saccharide was dissolved in THF (0.03M), NaHCO₃ solution (1M), 3 equiv., and Disuccinimidyl Carbonate, 1.5 equiv., was added at RT. Reaction mixture was heated at 45-50° C. for 30 min. TLC revealed that the reaction was over within 30 min. After extraction with Ethyl Acetate/dIH₂O. The Ethyl acetate was evaporated, and the residue was purified by flash chromatography over Silica Gel column.

Reaction of Saccharide Isocyanates with Diamines: Saccharide derivative was dissolved in THF (0.05M) and to the solution was added 0.6 equiv. of the diamine (0.1M). Reaction was stirred at RT for 1 h after which in most cases a white precipitate formed and was filtered as a white solid. White solid was washed with 20% hexanes-Ethyl Acetate and dried in oven to afford quantitative yields of the product.

Reaction of saccharides-Amine with t-Boc protected amino acid NHS-ester (linker): Amino sugar was dissolved in THF (0.1M) and NaHCO₃ (1M, 1.3 equiv.) and reagent (the linker) 1.4 equiv. was added at RT. Reaction mixture was heated 50° C. for 2 h, followed by stirring at RT overnight, product was extracted and purified on silica gel to afford >80% isolated yield.

Removal of 1-Boc (from linker-amine) saccharide with HCl/MeOH (3N): t-Boc derivative of saccharide was treated with HCl (3N)/MeOH: 1/10 solution overnight at RT. Methanol was evaporated and residue extracted with 0.05N NaOH/EtOAc. Product was purified by Silica Gel flash chromatography.

Global Deacetylation Reaction of saccharide: Acetylated Sugar, 0.05M solution in MeOH, was added NaOMe (25 wt. % in MeOH, 0.15 equiv.). Solution was stirred at RT. Reaction was completed within 2 h by TLC examination). Reaction mixture was concentrated and extracted with Ethyl acetate/Sat. Ammonium Chloride. product was purified on Silica Gel column.

Suberate Reaction: Amino sugar (such as compound 14, 1 equiv.) was dissolved in DMF (0.05 M). Suberate disuccinimidyl (0.035 equiv.) was dissolved in DMF (1M) and added to the Amino sugar solution. The mixture was heated to 45° C. for 4 h and then stirred at RT overnight. The reaction mixture was evaporated to the dryness and the product was extracted with Ethyl Acetate/dIH₂O. Product (such as compound 38) was isolated and purified on silica gel column.

General Procedure for O-sulfonation of saccharides: Saccharide (0.05 M solution in DMF/pyridine 1/1) was treated with Py:SO₃ complex (5 equiv./OH, added in 5 portions in 5 h) at 50° C. After completion of the addition, the mixture allowed to stir at RT overnight. The reaction mixture was evaporated to dryness and co-evaporated 3× with toluene to a pyridine-free residue. The residue was purified on C-18 column (reverse-phase bulk packing Dianion resin, styrenic Adsorbent, Sorbent Technology, 75-150 μm) or strong anion exchange column Macro Prep, Hi Q resin, Bio-Rad) followed by de-salting on (Sephadex G-25, Sigma-Aldrich).

General procedure for N-Sulfation of Amino Saccharides: To the amino saccharide (0.005M solution in DMF or DI H₂O) was added Na₂CO₃ (1M, 2 equiv./NH₃, to adjust the pH to 9-10). To the solution was added SO₃.Py or Me₃N:SO₃ complex (5 equiv./NH₂) at 50° C. for 2 h in 3 portions while maintaining the pH between 9-10. The reaction was stirred at RT for additional 8 h-18 h. Reaction mixture was concentrated, and the residue was fractionated on anion exchange resins (Macro Prep, Hi Q resin, Bio-Rad) using NaCl solution gradient (0-2.0 M). Fractions that were stained blue by the molybdate stain solution were collected, concentrated and de-salted on Size Exclusion column (Sephadex G-25, Sigma-Aldrich).

The following examples illustrate specific embodiments and utilities of the invention; they are not meant to limit the invention. As would be apparent to skilled artisans, various modifications in the composition, operation, and method are possible, and are contemplated herein without departing from the scope of the invention as defined in the claims.

### Example 1

### Preparation of Compound 12.

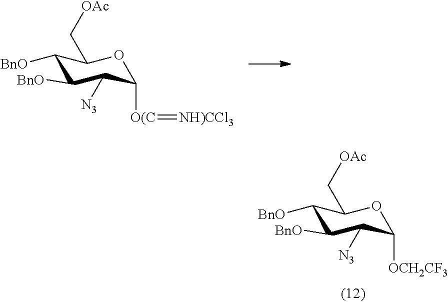

Trichloroacetimidate derivative (compound 11) and Trifluoroethanol were dissolved in methylene chloride at room temperature and dried by co-evaporating with Toluene (3×10 mL). Dried material was dissolved in 25 mL Dichloromethane and at room temperature triethylsilyl triflate was added. The reaction mixture was worked up after 3 h and purified on silica gel column. Compound 12, Yield: 1.9 g, 98%.

### Example 2

### Preparation of Compound 13.

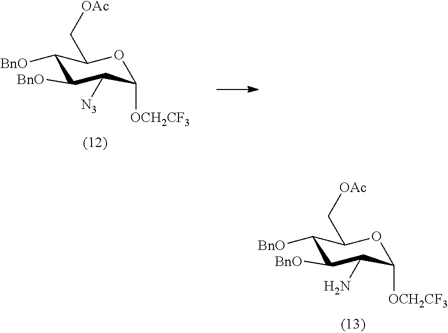

Monosaccharide (12) was dissolved in Ethanol and vigorously stirred at Room Temperature. Zinc powder was added slowly. Ammonium chloride solution was added dropwise in 1 h. After completion of addition, the mixture was allowed to stir overnight. The reaction mixture was filtered through thin pad of Silica Gel and evaporated to dryness. The white residue was extracted with EtOAc/H2O, dried over Na₂SO₄ and evaporated to white foam. Compound 13, Yield: 1.64 g/85% (Crude).

### Example 3

### Preparation of Compound 14.

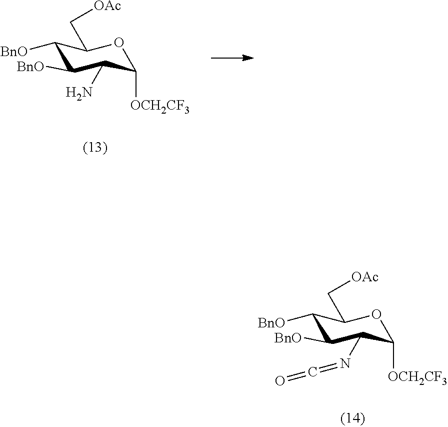

Monosaccharide (13) was dissolved in THE, NaHCO₃ solution was added. Reagent was added at room temperature. The reaction mixture was heated to 45° C. Reaction was completed in 30 min. as judged by TLC. Yield after flash chromatography: Compound 14, 344 mg (94%).

### Example 4

### Preparation of Compound 15.

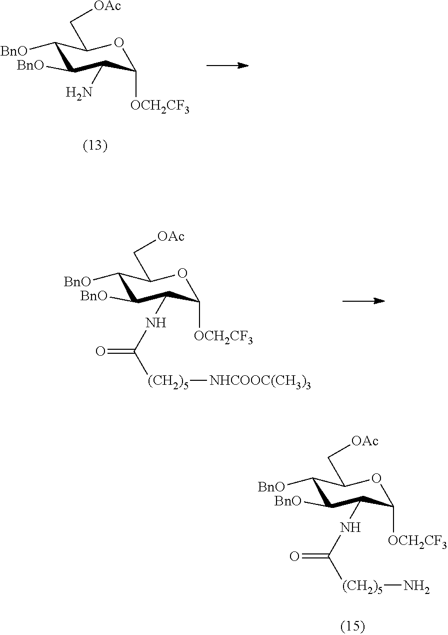

Monosaccharide (14) was dissolved in THF. Sodium bicarbonate solution added. The reagent was added at RT and the mixture was refluxed for 1 h. The reaction mixture was stirred at RT overnight. The product was purified on Silica Gel. Yield (81%). The purified t-Boc derivative was treated with HCl/MeOH overnight at RT. Methanol was evaporated ant the residue was purified on Silica gel purified material. Yield: (78%, over two steps)

### Example 5

### Preparation of Compound 15A.

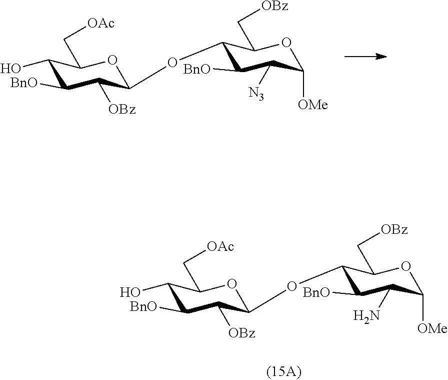

of BA-Dimer was dissolved in 15 mL THF. Clear colorless solution. Reaction solution was heated to 50° C. Triphenylphosphine, 368 mg, was added in one portion at 50° C. Clear colorless solution. The mixture was monitored by TLC (40% E-H & 100% E). After 5 h, reaction mixture was cooled down to room temperature. 3 mL H₂O was added, and the mixture was stirred at room temperature overnight. TLC before and after 30 min addition of H₂O. Next day, 10 mL HCl 2N was added to the reaction mixture followed by 15 mL EtOAc. EtOAc layer was separated, dried, evaporated and charged as DCM solution on Silica Gel Column (Elution: 50 mL each, EtOAc-H 40, 80, 100%, 2×0.5% MeOH-E, 10%, 20% MeOH-E). Fractions containing the major product were collected, evaporated to a white solid. Compound 15A, Yield: 400 mg (74%).

### Example 6

### Preparation of Compound 15B.

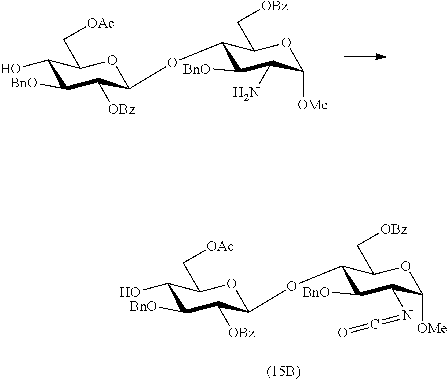

Disaccharide (15) was dissolved in THF. dIH₂O was added. Reagent was added at RT. The reaction solution was heated to 50° C. Reaction was monitored by LC (15% M-E & 60% E-H). TLC after 1 h showed completion of reaction. Flash Chromatography (40%-70% EtOAc-Hexanes) afforded: Compound 15B, 1.45 g (98%).

### Example 7

### Preparation of Compound 16 and 17.

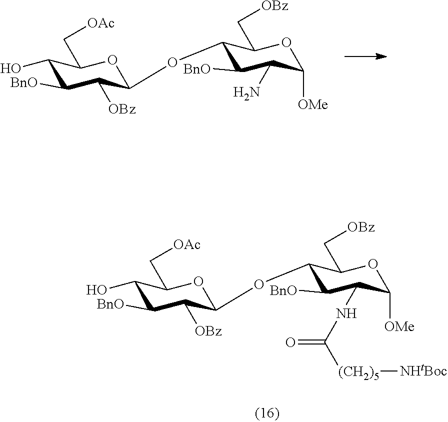

t-Boc Reagent and disaccharide BA (2) dissolved in THF, 2 mL 1M NaHCO₃ and 2 mL of diH2O were added. TLC examination showed completion of the reaction >90%. After Flash Chromatography, Compound 16, Yields: 400 mg, 81%.

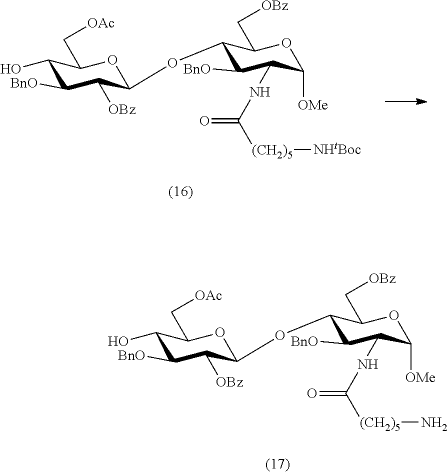

Compound 16 dissolved in Methanolic HCl (3N) and the mixture stirred at RT overnight. Methanol was removed by evaporation and product was purified by Silica Gel, Compound 17, Yield: (87%), 920 mg.

### Example 8

### Preparation of Compound 18.

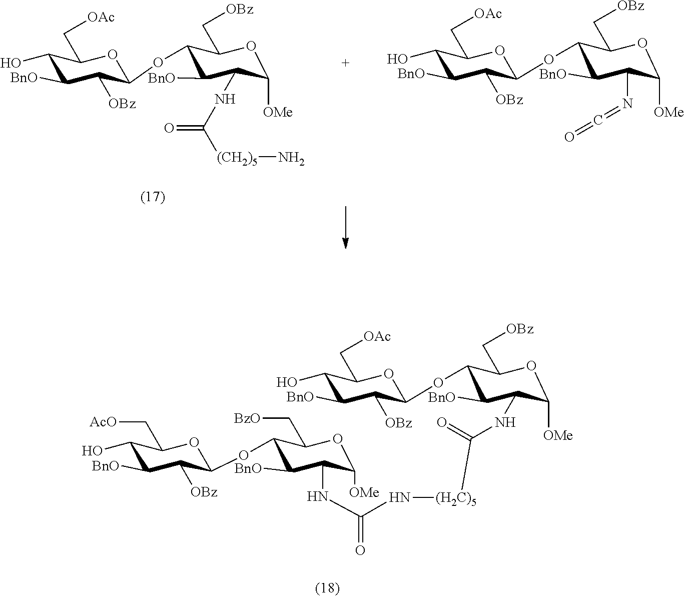

Disaccharide (15) was dissolved in THF. Sat. Bicarbonate solution was added. Di(N-Succinimidyl) Suberate was then added. Mixture was heated to 60° C. for 2 h, and then stirred at room temperature or 6 h. Product was extracted and separated on silica Gel Column. Compound 18, Yields: (89%)

### Example 9

### Preparation of Compounds 19, 20, and 21.

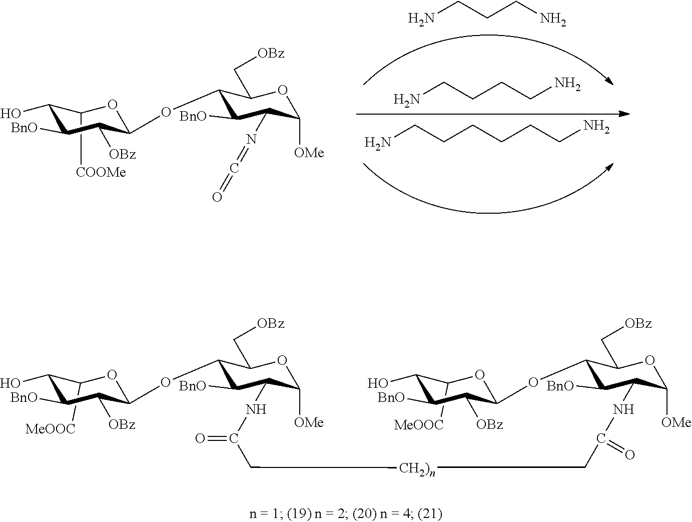

General Procedure: Disaccharide (15A) derivative was dissolved in THF and diamine in THF (5 mL) prepared and added. After stirring at room temperature for 1 h, the white precipitate was filtered as white solid. The white solid was washed with 20% hexanes-ethyl acetate and dried. The yield is quantitative.

### Example 10

### Preparation of Compound 22.

### Step 1

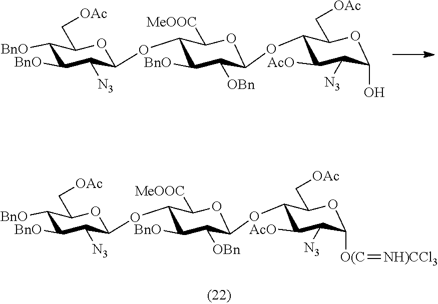

Trisaccharide (5) was dried by dissolving in 25 mL dry toluene and evaporation to dryness (white foam) three times. Dried trisaccharide was dissolved in dry DCM (65 mL). the solution was cooled to 0° C. in an ice bath. Trichloroacetonitrile was added followed by addition of DBU. The mixture was stirred and allowed to warm up to room temperature overnight. Reaction mixture was extracted by dilute NaHCO₃ aq. Solution, dried over Na₂SO₄ and evaporated to dryness and kept under high vacuum overnight and without further purification was taken to the next step. Crude Yield: 4.2 g. The Dry Acetimidate derivative was dissolved in dry methylene chloride at room temperature. Trifluoroethanol was added. At room temperature, To the reaction mixture was added TESOTf. The mixture was stirred for 2 hours at room temperature. The reaction mixture was extracted with dIH2O and purified on Silica gel column. Compound 22, Yield: 3.6 g (90%). Note: Rf of the product is slightly higher that the acetimidate derivative.

### Example 11

### Preparation of Compound 23.

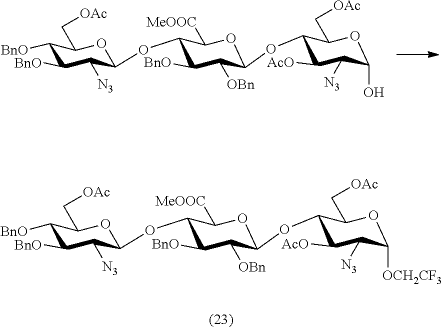

Trisaccharide (23) 1 mmol, 1 g, was dissolved in EtOH, 100 mL at room temperature. Zinc powder, 16 mmol 1 g. was added, and the mixture was cooled in an ice bath. Ammonium chloride solution 24 mmol was added drop-wise in 15 min. Ice bath was removed, and the reaction mixture was stirred vigorously at room temperature overnight. Reaction mixture after overnight stirring at room temperature (White-gray suspension liquid) was evaporated to dryness. The white residue was extracted first with 100 mL EtOAc/dIH₂O) and then the aqueous layer was treated with 25 mL NaOH, 0.5N, and extracted one more time with EtOAc. TLC examination showed that major product is in the first EtOAc layer and the second EtOAc had minute amount of the product as judged by Molybdate staining. The combined EtOAc layers was dried (Na₂SO₄) and evaporated to a white Foam. Compound 23i (710 mg, 67%). Product was purified on silica gel column using acetonitrile-dIH₂O (gradient 0-10%).

### Example 12

### Preparation of Compound 23i.

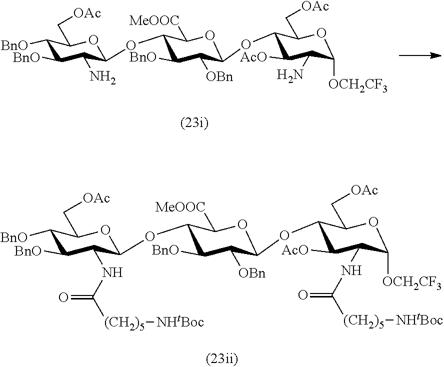

Trisaccharide derivative 23i, 700 mg, was dissolved in THF 35 mL at room temperature. Aq. Solution of NaHCO₃, 2 mL, was added and the mixture was heated to 50° C. N-hydroxy succinimide derivative 16 mmol added and the heating was continued for two hours and then allowed the mixture to be stirred overnight. Reaction mixture was extracted and flashed to afford Compound 23ii, 280 mg (29%).

### Example 13

### Preparation of Compound 24.

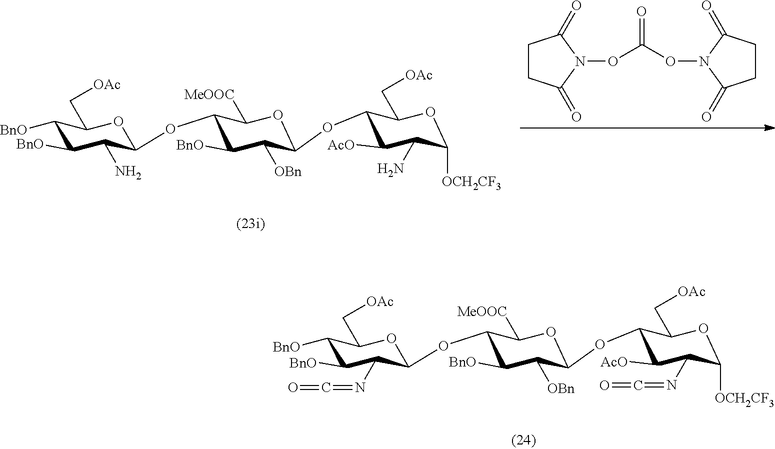

Trisaccharide 23i, 0.1 mmol and Carbonate derivative 0.3 mmol were dissolved in 10 mL THF. dIH₂O (1 mL) was added. The mixture was heated to 50° C. for 3 h. TLC showed the completion of the reaction. Mass analysis indicated the formation of bis-NHS derivative is almost instantly. Reaction was allowed stir at room temperature for total 3 h. Reaction mixture extracted and purified on silica gel column to afford the product. Compound 24, Yield (500 mg scale): 350 mg (79%)

### Example 14

### Preparation of Compound 25.

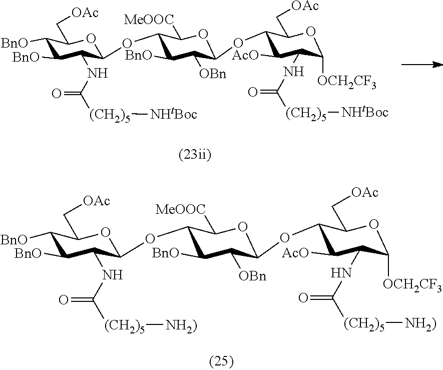

Intermediate compound (23ii), 3.1 g (10 mmol), was treated with Methanolic HCl solution (3N, 10 mL) at room temperature for 18 h. The reaction mixture was evaporated and extracted with EtOAc and 1N Na₂CO₃ (1:1, 3×30 mL). Yield (25): 2.5 g (84%).

### Example 15

### Preparation of Compound 26.

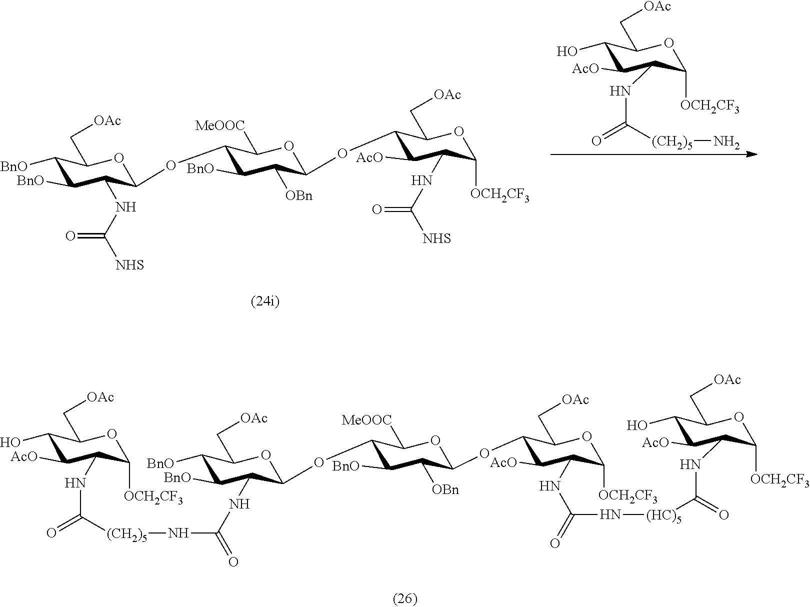

Both reactants were dried by co-evaporation from Toluene (3×1 mL). The dried materials dissolve in dry THF (12 mL). The mixture was stirred at room temperature. TLC after 30 min showed completion of the reaction. Extraction and chromatography purification delivered the desired product, Compound 26, quantitative.

### Example 16

### Preparation of Compound 27.

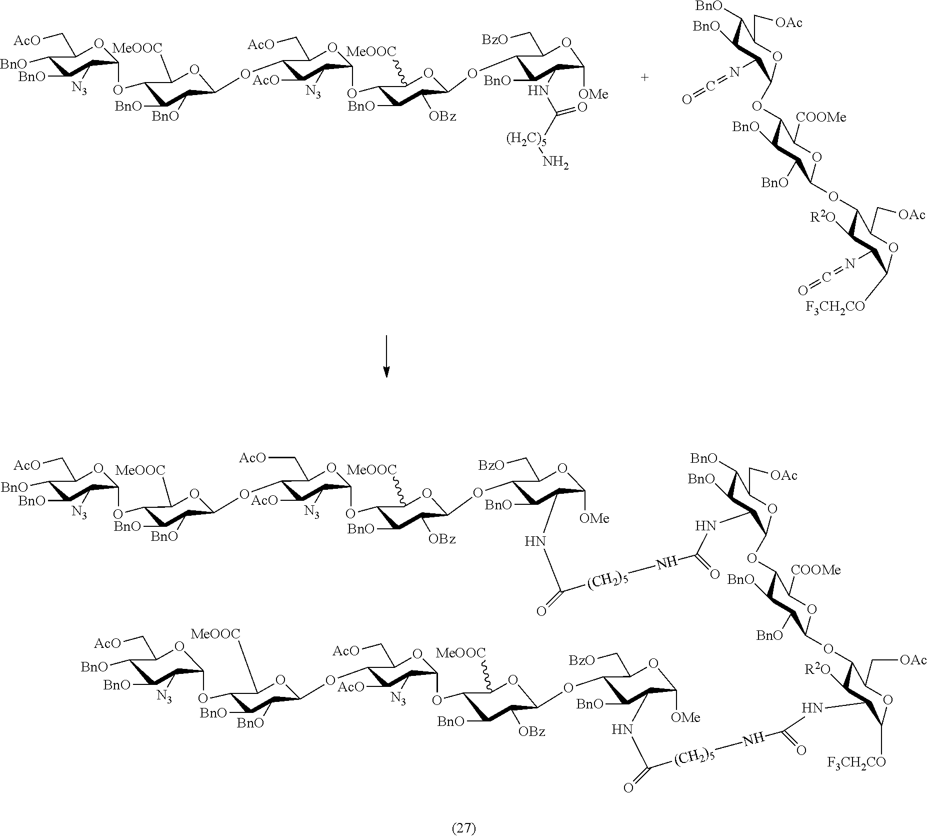

The reactants (Compound 24 and 30) were dissolved in 10 mL THF. 100 mL of NaHCO₃ (1M) was added. The mixture was heated to 50° C. for 2 h. Flash chromatography produced the desired product, Compound 27 (quantitative).

### REFERENCES

Avci, F., Karst, N., & Linhardt, R. (2003). Synthetic Oligosaccharides as Heparin-Mimetics Displaying Anticoagulant Properties. Current Pharmaceutical Design, 9(28), 2323-2335. https://doi.org/10.2174/1381612033453929
Billings, P. C., Yang, E., Mundy, C., & Pacifici, M. (2018). Domains with highest heparan sulfate-binding affinity reside at opposite ends in BMP2/4 versus BMP5/6/7: Implications for function. Journal of Biological Chemistry, 293(37), 14371-14383. https://doi.org/10.1074/jbc.RA118.003191
Dey, S., Lo, H.-J., & Wong, C.-H. (2020). Programmable One-Pot Synthesis of Heparin Pentasaccharide Fondaparinux. Organic Letters, 22(12), 4638-4642. https://doi.org/10.1021/acs.orglett.0c01386
Hansen, S. U., Miller, G. J., Cole, C., Rushton, G., Avizienyte, E., Jayson, G. C., & Gardiner, J. M. (2013). Tetrasaccharide iteration synthesis of a heparin-like dodecasaccharide and radiolabelling for in vivo tissue distribution studies. Nature Communications, 4(1), 2016. https://doi.org/10.1038/ncomms3016
Horton, M., Su, G., Yi, L., Wang, Z., Xu, Y., Pagadala, V., Zhang, F., Zaharoff, D. A., Pearce, K., Linhardt, R. J., & Liu, J. (2021a). Construction of heparan sulfate microarray for investigating the binding of specific saccharide sequences to proteins. Glycobiology, 31(3), 188-199. https://doi.org/10.1093/glycob/cwaa068
Horton, M., Su, G., Yi, L., Wang, Z., Xu, Y., Pagadala, V., Zhang, F., Zaharoff, D. A., Pearce, K., Linhardt, R. J., & Liu, J. (2021b). Construction of heparan sulfate microarray for investigating the binding of specific saccharide sequences to proteins. Glycobiology, 31(3), 188-199. https://doi.org/10.1093/glycob/cwaa068
Leeuwenburgh, M. (2003). Olefin metathesis in glycobiology: new routes towards diverse neoglycoconjugates. Current Opinion in Chemical Biology, 7(6), 757-765. https://doi.org/10.1016/j.cbpa.2003.10.009
Li, J.-P., & Kusche-Gullberg, M. (2016). Heparan Sulfate: Biosynthesis, Structure, and Function (pp. 215-273). https://doi.org/10.1016/bs.ircmb.2016.02.009
Liao, Y.-E., Xu, Y., Arnold, K., Zhang, F., Li, J., Sellers, R., Yin, C., Pagadala, V., Inman, A. M., Linhardt, R. J., Xu, D., Pawlinski, R., & Liu, J. (2023). Using heparan sulfate octadecasaccharide (18-mer) as a multi-target agent to protect against sepsis. Proceedings of the National Academy of Sciences, 120(4). https://doi.org/10.1073/pnas.2209528120
Ma, S.-N., Mao, Z.-X., Wu, Y., Liang, M.-X., Wang, D.-D., Chen, X., Chang, P., Zhang, W., & Tang, J.-H. (2020). The anti-cancer properties of heparin and its derivatives: a review and prospect. Cell Adhesion & Migration, 14(1). https://doi.org/10.1080/19336918.2020.1767489
Mulloy, B. (2019). The non-anticoagulant promise of heparin and its mimetics. Current Opinion in Pharmacology, 46. https://doi.org/10.1016/j.coph.2019.03.009
Multhaupt, H. A. B., & Couchman, J. R. (2012). Heparan Sulfate Biosynthesis. Journal of Histochemistry & Cytochemistry, 60(12), 908-915. https://doi.org/10.1369/0022155412460056
Nugent, M. A., Zaia, J., & Spencer, J. L. (2013). Heparan sulfate-protein binding specificity. Biochemistry (Moscow), 78(7), 726-735. https://doi.org/10.1134/S0006297913070055
Stopschinski, B. E., Thomas, T. L., Nadji, S., Darvish, E., Fan, L., Holmes, B. B., Modi, A. R., Finnell, J. G., Kashmer, O. M., Estill-Terpack, S., Mirbaha, H., Luu, H. S., & Diamond, M. I. (2020). A synthetic heparinoid blocks Tau aggregate cell uptake and amplification. Journal of Biological Chemistry, 295(10). https://doi.org/10.1074/jbc.RA119.010353
Tran, V. M., Wade, A., Mckinney, A., Chen, K., Lindberg, O. R., Engler, J. R., Persson, A. I., & Phillips, J. J. (2017). Heparan Sulfate Glycosaminoglycans in Glioblastoma Promote Tumor Invasion. Molecular Cancer Research, 15(11). https://doi.org/10.1158/1541-7786.MCR-17-0352
Weiss, R. J., Esko, J. D., & Tor, Y. (2017). Targeting heparin and heparan sulfate protein interactions. Organic & Biomolecular Chemistry, 15(27). https://doi.org/10.1039/C7OB01058C
Xu, D., & Esko, J. D. (2014). Demystifying Heparan Sulfate—Protein Interactions. Annual Review of Biochemistry, 83(1). https://doi.org/10.1146/annurev-biochem-060713-035314
Yang, C., Deng, Y., Wang, Y., Xia, C., Mehta, A. Y., Baker, K. J., Samal, A., Booneimsri, P., Lertmaneedang, C., Hwang, S., Flynn, J. P., Cao, M., Liu, C., Zhu, A. C., Cummings, R. D., Lin, C., Mohanty, U., & Niu, J. (2023). Heparan sulfate glycomimetics via iterative assembly of “clickable” disaccharides. Chemical Science, 14(13), 3514-3522. https://doi.org/10.1039/D3SC00260H
Zhang, J., Liang, L., Yang, W., Ramadan, S., Baryal, K., Huo, C., Bernard, J. J., Liu, J., Hsieh-Wilson, L., Zhang, F., Linhardt, R. J., & Huang, X. (2022). Expedient Synthesis of a Library of Heparan Sulfate-Like “Head-to-Tail” Linked Multimers for Structure and Activity Relationship Studies **. Angewandte Chemie International Edition, 61(48). https://doi.org/10.1002/anie.202209730

We claim:

1. A composition consisting of spaced heparinoids of Formulas 1 and 2, 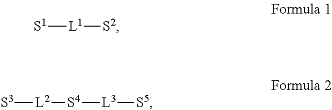
wherein
S¹, S², S³, S⁴, and S⁵ are independently selected from the group comprising: 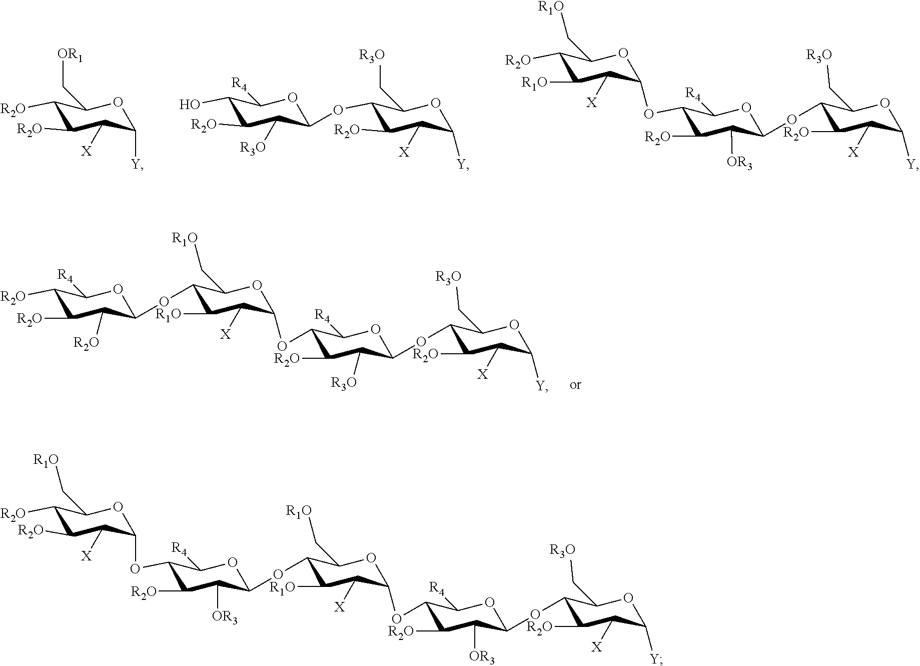
wherein L¹, L², and L³ are independently selected from the group consisting of —CO(CH₂)ₐCO—; —CO(CH₂)_{b}NHCO—; —CONH(CH₂)_{c}CO—; —CONH(CH₂)_{d}NHCO—; —CO(CH₂)ₑNHCO(CH₂)_{f}(CONH(CH₂)_{g}CO—; X is —N₃, —NH—, —NH₂, —NHAc, —NHSO₃Na, or —NCO; Y is —OH, —OMe, —OCH₂CF₃, or C₅-C₁₀-OAryl; R¹ is —H, —CH₂OH or C₁-C₁₀ Alkyl; R² is Bn or PMB; R³ is C₁-C₁₀ Acyl or Bz; and R⁴ is —COOH or —COONa; and subscripts ‘a’, ‘b’, ‘c’, ‘d’, ‘e’, ‘f’, and ‘g’ independently vary from 1-10.

2. A composition of claim 1 comprising Type 1+1 structure 1 of Formula 1, 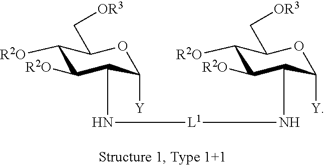
wherein L¹ is —CO(CH₂)ₐCO—.

3. A composition of claim 2, wherein Y is —OH; R¹, R², and R³ are independently —H or —SO₃Na; and subscript ‘a’ varies from 4-6.

4. A composition of claim 1 comprising Type 1+2 structure 2 of Formula 1, 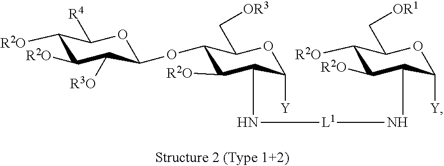
wherein L¹ is —CO(CH₂)ₐCO— or —CO(CH₂)_{b}NHCO—.

5. A composition of claim 4, wherein Y is —OH; R¹, R², and R³ are independently —H or —SO₃Na; R⁴is —COONa; and each of the subscripts ‘b’ and ‘c’ independently vary from 4-6.

6. A composition of claim 1 comprising Type 2+2 structure 3 of Formula 1, 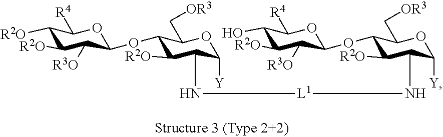
wherein L¹ is —CO(CH₂)ₐCO—, —CO(CH₂)_{b}NHCO—, or —CONH(CH₂)_{c}CO—.

7. A composition of claim 6, wherein Y is —OH; R¹, R², and R³ are independently —H or —SO₃Na; R⁴is —COONa; and each of the subscripts ‘a’, ‘b’, and ‘c’ independently vary from 4-6.

8. A composition of claim 1 comprising Type 1+3+1 structure 4 of Formula 2, 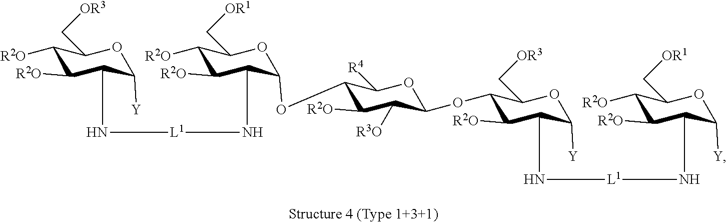
wherein L¹ is —CO(CH₂)ₐCO—, —CO(CH₂)_{b}NHCO—, or —CONH(CH₂)_{c}CO—.

9. A composition of claim 8, wherein Y is —OH; R¹, R², and R³ are independently —H or —SO₃Na; R⁴ is —COONa; and each of the subscripts ‘a’, ‘b’, and ‘c’ independently vary from 4-6.

10. A composition of claim 1 comprising Type 2+3+2 structure 5 Formula 2, 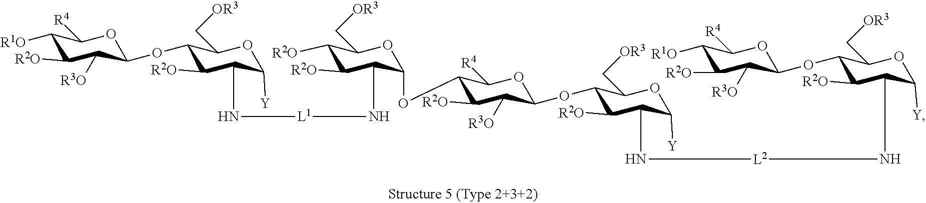
wherein L¹ and L² are independently —CO(CH₂)_{b}NHCO—.

11. A composition of claim 10, wherein Y is —OH; R¹, R², and R³ are independently —H or —SO₃Na; R⁴ is —COONa; and subscript ‘b’ varies from 4-6.

12. A composition of claim 1comprising Type 4+4 structure 6 of Formula 1, 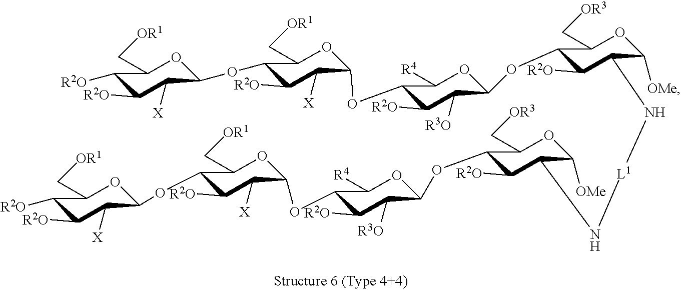
wherein L¹ is —CO(CH₂)ₐCO—, —CO(CH₂)_{b}NHCO—, or —CONH(CH₂)_{c}CO—

13. A composition of claim 12, wherein R¹, R², and R³ are independently —H or —SO₃Na; R⁴ is —COONa; and each of the subscripts ‘a’, ‘b’, and ‘c’ independently vary from 4-6.

14. A composition of claim 1 comprising Type 5+5 structure 7 of Formula 1, 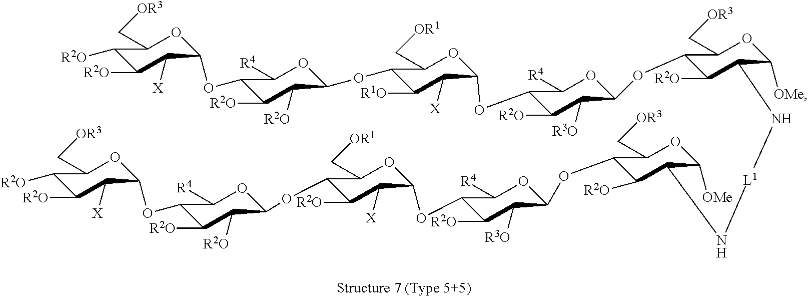
wherein L¹ is —CO(CH₂)ₐCO—, —CO(CH₂)_{b}NHCO—, or —CONH(CH₂)_{c}CO—.

15. A composition of claim 14, wherein R¹, R², and R³ are independently —H or —SO₃Na; R⁴ is —COONa; and each of the subscripts ‘a’, ‘b’, and ‘c’ independently vary from 4-6.

16. A composition of claim 1 comprising Type 5+5 structure 8 of Formula 1, 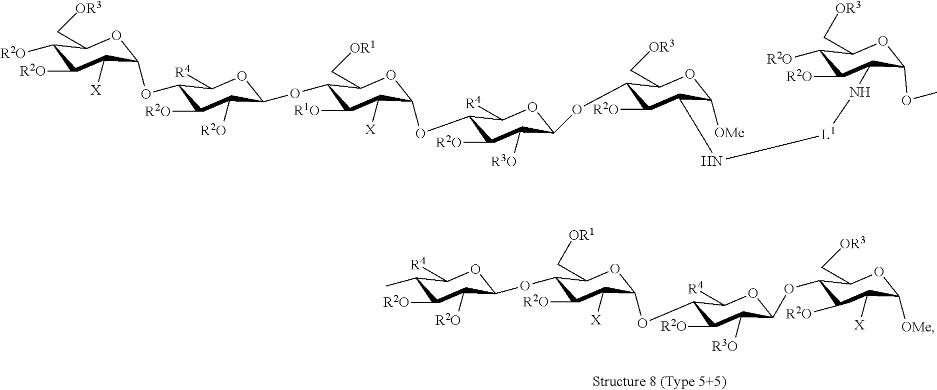
wherein L¹ is —CO(CH₂)ₐCO—, —CO(CH₂)_{b}NHCO—, or —CONH(CH₂)_{c}CO—.

17. A composition of claim 16, wherein R¹, R², and R³ are independently —H or —SO₃Na; R⁴ is —COONa; and each of the subscripts ‘a’, ‘b’, and ‘c’ independently vary from 4-6.

18. A composition of claim 1 comprising Type 5+3+5 structure 9 of Formula 2, 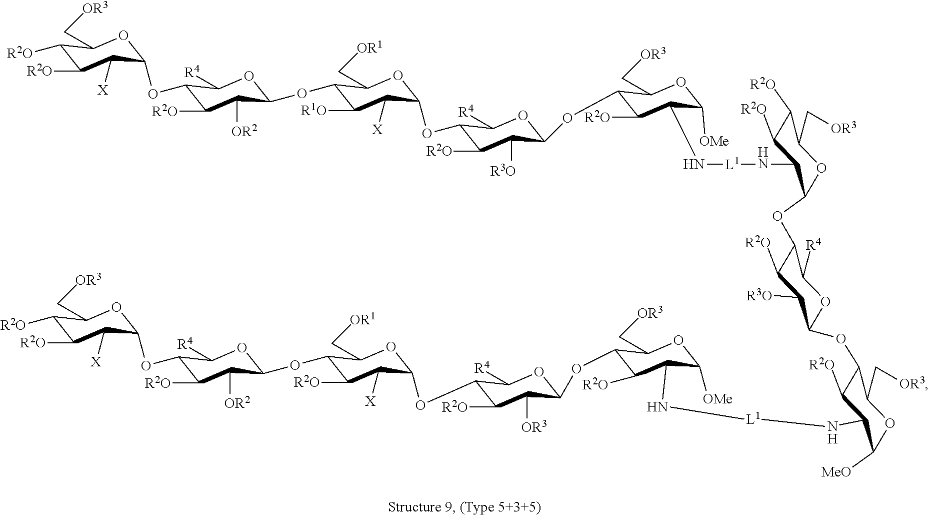
wherein L¹ is —CO(CH₂)ₐCO— or —CO(CH₂)_{b}NHCO—.

19. A composition of claim 18, wherein R¹, R², and R³ are independently —H or —SO₃Na; R⁴ is —COONa; and each of the subscripts ‘b’ and ‘c’ independently vary from 4-6.

20. A process for the preparation of spaced heparinoids of Formulas 1 and 2, 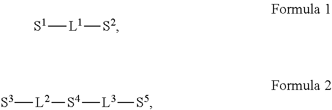
wherein
S¹, S², S³, S⁴, and S⁵ are independently selected from the group comprising: 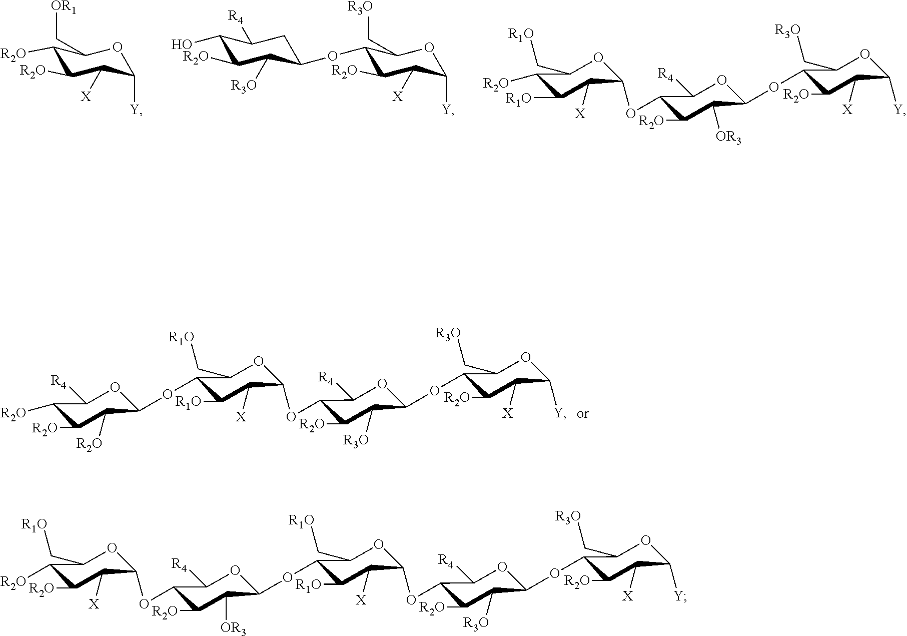
wherein L¹, L², and L³ are independently selected from the group consisting of —CO(CH₂)ₐCO—; —CO(CH₂)_{b}NHCO—; —CONH(CH₂)_{c}CO—; —CONH(CH₂)_{d}NHCO—; —CO(CH₂)ₑNHCO(CH₂)_{f}CONH(CH₂)_{g}CO—; X is —N₃, —NH—, —NH₂, —NHAc, —NHSO₃Na, or —NCO; Y is —OH, —OMe, —OCH₂CF₃, or C₅-C₁₀-OAryl; R¹ is —H, —CH₂OH or C₁-C₁₀ Alkyl; R² is Bn or PMB; R³ is C₁-C₁₀ Acyl or Bz; and R⁴ is —COOH or —COONa; and subscripts ‘a’, ‘b’, ‘c’, ‘d’, ‘e’, ‘f’, and ‘g’ independently vary from 1-10, and wherein the steps comprising:
(a) preparing the trichloroacetimidates of azidosaccharides as described in paragraph [0032];
(b) glycosylating the acetimidate derivatives of the azidosaccharides in Step ‘a’ as described in the specification, paragraph [0032];
(c) reducing the azide in Step ‘b’ to the corresponding mine using either Zn/ammonium chloride or triphenylphosphine as described in paragraphs [0034] or [0035] respectively;
(d) reacting the amino sugars in Step ‘c’ with disuccinimidyl carbonate as described in paragraph; [0036];
(e) reacting the saccharide isocyanates in Step ‘d’ with diamines as described in paragraph; [0037];
(f) reacting the saccharides-amine in Step ‘e’ with t-Boc protected amino acid NHS-ester (linker) as described in paragraph [0038];
(g) removing the t-Boc protected saccharides in Step ‘f’ with 3N HCl/MeOH as described in paragraph [0039];
(h) globally deacetylating the saccharides in Step ‘g’ with NaOMe/MeOH as described in paragraph [0040];
(i) reacting the amino sugars in Step ‘i’ with disuccinimidyl suberate as described in paragraph; [0041]; and
(j) O-sulfonating or N-sulfonating the oligosaccharides in Step ‘i’ with pyrdine-sulfur trioxide complex as described in paragraphs [0042] and [0043] respectively.
